(19) United States
(12) Patent Application Publication
(10) Pub. No.: US 2025/0249238 A1
(43) Pub. Date: Aug. 7, 2025

(54) LEAD EXTENDER FOR EXTENDING LEADS FOR EXTRACTION

(71) Applicant: Medtronic, Inc., Minneapolis, MN (US)

(72) Inventors: Mikayle A. Holm, Minneapolis, MN (US); Kevin Seifert, Forest Lake, MN (US); Mark T. Marshall, Cape Coral, FL (US)

(21) Appl. No.: 18/856,816

(22) PCT Filed: Apr. 27, 2023
(86) PCT No.: PCT/US2023/066312

§ 371 Date: Oct. 14, 2024

Related U.S. Application Data
(60) Provisional application No. 63/363,685, filed on Apr. 27, 2022.

(51) Int. Cl. A61N 1/05; A61B 17/34

(52) U.S. Cl. CPC A61N 1/057; A61B 17/3468; A61N 2001/0578

(57) ABSTRACT

A lead extender comprises a connector defining a chamber having an inner wall and an opening into the chamber, the chamber configured to receive a proximal portion of a cut implantable medical lead through the opening. The connector comprises a locking mechanism configured to lock the proximal portion of the cut implantable medical lead to the inner wall of the chamber. The lead extender further comprises an extension line, having a distal end attached to the connector, the extension line configured to extend through a lumen of an extraction tool to the proximal end controlled by the operator for extracting the cut implantable medical lead from a patient.

This application claims the benefit of U.S. Provisional Patent Application Ser. No. 63/363,685, filed Apr. 27, 2023, the entire content of which is incorporated herein by reference.

### TECHNICAL FIELD

The disclosure relates to medical devices and, more particularly to extraction of implantable medical leads.

### BACKGROUND

Implantable medical systems commonly include one or more implantable medical leads coupled to an implantable or external medical device to provide a therapy to the patient. As an example, cardiac systems, such as implantable pacemakers, cardioverter-defibrillators, cardiac resynchronization therapy devices and the like, commonly include an implantable medical device (IMD) such as an implantable pulse generator electrically connected to the heart by at least one transvenous endocardial lead. An endocardial lead provides an electrical pathway between the pacemaker, connected to the proximal end of the lead, and endocardial tissue, which is in contact with the distal end of the lead. Endocardial tissue refers to a specific layer of tissue in the interior of the heart's chambers. Electrical stimulation pulses emitted by the IMD travel through the endocardial lead and stimulate the heart to deliver a prescribed therapy. Other implantable medical systems, such as neuromodulation stimulators, may have leads implanted in other locations of the patient, e.g., brain, spine and the like.

In some patients, it may become necessary to extract and replace an implanted lead. For example, a lead may need to be acutely replaced when unacceptable stimulation thresholds are measured during an implant procedure. A lead may also need to be replaced when the lead fails, or when the tissue around the lead implantation site becomes infected.

In some examples, if the lead has been implanted for an extended period of time, scar tissue formed around the lead can make the extraction procedure more difficult. In such cases, a tubular extraction sheath may be used to track over the length of the lead. The extraction sheath is guided within the patient to dissect the fibrotic scar adhesions that prevent lead extraction. In some examples, a distal tip of the tubular extraction sheath may include a rotating cutting element, a laser, a plasma generator, or an electrocautery system to assist in dissecting the adhesions. Once the tubular extraction sheath has been advanced to the implantation site, the lead may be extracted through the sheath and removed from the body of the patient.

In some examples, an inside diameter of an extraction sheath is about 7 French (Fr) (2.3 mm) to as large as 13 Fr (4.3 mm), and smaller extraction sheaths are desirable to prevent injury to the vasculature of the patient. However, the proximal end of pacemaker leads typically have a relatively bulky connector assembly that plugs into, and makes an electrical connection with, the pacemaker. Standard connectors used to connect leads to pacemakers, such as IS-1, IS-4 and DF-4, have an outside diameter of about 14 Fr to 15 Fr (4.6 mm to 5.0 mm). In some procedures, when extracting a lead, the relatively large diameter of the connector can require that the lead be cut near the connector to allow insertion of the lead into the extraction sheath.

### SUMMARY

Once cut, the lead requires additional preparation steps to bind the conductors to a suitable locking stylet or compression coil so that traction may be applied to the lead. Incorrect preparation of the severed conductors can decrease lead tensile strength and increase the chance of lead breakage. Some lead designs can lose substantial lead strength when cut, and cutting the lead to remove the bulky connector may require lead insulation and other protective layers overlying the lead conductor be immobilized by, for example, a ligation suture. Preparation of the lead can be time consuming, requiring patients to be under anesthesia for longer periods of time. Additionally, incorrect preparation of the severed conductors can decrease the traction force the extraction tool can provide to the prepared lead.

Traction force applied during use of common mechanical extraction tools and techniques is often limited by the size of the mechanism providing the traction force. The size of the mechanism is particularly limited by the inner diameter of the extraction sheath. Maintaining a narrow sheath diameter and therefore a thin extraction tool is important to prevent vascular damage during extraction of the lead. However, maintaining traction on the lead with a smaller tool may be difficult.

In general, the present disclosure is directed to techniques that include application of a lead extender to a prepared lead for purposes of extraction. The lead extender of the present disclosure includes a connector that attaches an extension wire or line to a proximal portion of a prepared cut lead. The connector includes a locking mechanism. The connection created by the locking mechanism is capable of withstanding enough tensile strength for the operator to apply tension to the tissue, e.g., endocardial tissue, at the site of the lead's connection to the tissue via the lead. The operator may apply the traction force to the lead via the extension line extending through a lumen of an extraction sheath during advancement of the sheath over the lead. In this manner, the techniques of the present disclosure may advantageously provide adequate traction for extraction of a lead via an extraction sheath with an appropriately sized tool.

In one example, a lead extender including a connector defining a chamber having an inner wall and an opening into the chamber, the chamber configured to receive a proximal portion of a cut implantable medical lead through the opening, wherein the connector comprises a locking mechanism configured to lock the proximal portion of the cut implantable medical lead to the inner wall of the chamber. The lead extender also includes an extension line, having a distal end attached to the connector, the extension line configured to extend through a lumen of an extraction tool from a distal end to a proximal end of the extraction tool.

In another example, a method including receiving, by a connector defining a chamber having an inner wall and an opening into the chamber, a proximal portion of a cut implantable medical lead through the opening, wherein the connector comprises a locking mechanism configured to lock the proximal portion of the cut implantable medical lead to the inner wall of the chamber. The method also including extending, through a lumen of an extraction tool from a distal end to a proximal end of the extraction tool, an extension line attached to the connector via a distal end.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1 is a conceptual drawing illustrating an example system that includes a temporary or permanent implantable medical device (IMD) coupled to an implantable medical lead.

FIGS. 2A-2E illustrate example cut implantable medical leads.

FIGS. 3A-3C are conceptual diagrams illustrating an example lead extender.

FIGS. 3D and 3E illustrate example configurations of anchor posts that may be used with a lead extender as illustrated in FIGS. 3A-3C, according to this disclosure.

FIG. 3F illustrates an example configuration of a lead extender without an extension line, according to this disclosure.

FIG. 3G illustrates an example a deformable sleeve, according to this disclosure.

FIGS. 4A and 4B are conceptual diagrams illustrating another example lead extender, according to this disclosure.

FIGS. 5A and 5B are conceptual diagrams illustrating another example lead extender, according to this disclosure.

FIG. 6 is a flow diagram illustrating an example method for using a lead extender, in accordance with one or more techniques of this disclosure.

FIG. 7 is a flow diagram illustrating an example method for extracting a cut implantable medical lead using a lead extender according to this disclosure.

### DETAILED DESCRIPTION

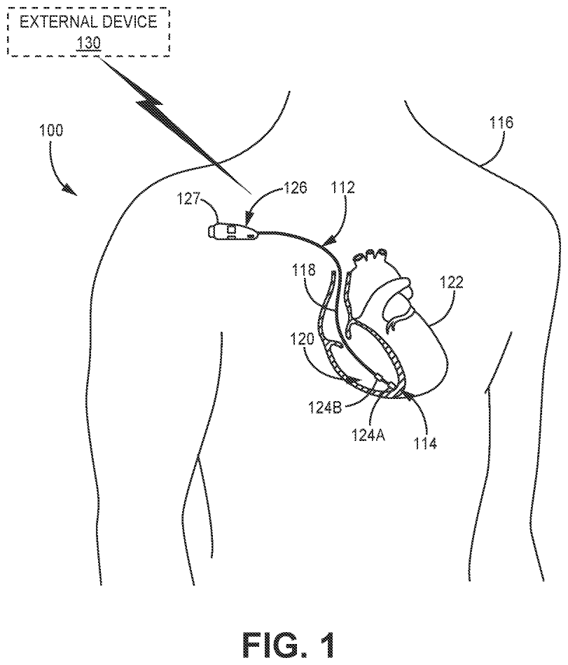

FIG. 1 is a conceptual diagram illustrating a portion of an example implantable medical device system 100. As illustrated by the example of FIG. 1, implantable medical device system 100 may function as a single chamber, e.g., ventricular, pacemaker that delivers pacing to a heart 122 of patient 116. Implantable medical device system 100 may be a temporary or permanent pacemaker. In alternative embodiments, however, implantable medical device system 100 may include one or more leads and function as a multi-chamber pacemaker, such as a dual-chamber pacemaker or triple-chamber pacemaker that delivers pacing to a heart 122 of patient 116. In some examples, the devices and methods of the present disclosure may be implemented in IMDs other than pacemakers, such as implantable cardioverter-defibrillators (ICDs), implantable cardiac resynchronization (CRT) devices, implantable neurostimulators, or other implantable medical systems that couple to implanted medical leads.

In the example of FIG. 1, implantable medical device system 100 includes one or more implantable medical leads 112 electrically connected to the IMD 126. The implantable medical lead 112 includes an elongated lead body 118 with a distal portion 120 positioned at a target implantation site 114 within the heart 122 such as, for example, a wall within one or more ventricles or atria. The lead 112 may be a unipolar, a bipolar, or a multipolar lead.

A clinician may maneuver the distal portion 120 of the lead 112 through the vasculature of patient 116 to position the distal portion 120 at or near the target site 114. For example, the clinician may guide distal portion 120 through the superior vena cava (SVC) to target site 114 on or in a ventricular wall of heart 122, e.g., at the apex of the right ventricle as illustrated in FIG. 1. Implantable medical device system 100 may include a delivery catheter and/or outer member (not shown), and implantable medical lead 112 may be guided and/or maneuvered within a lumen of the delivery catheter in order to approach target site 114.

The implantable medical lead 112 includes one or a plurality of electrodes. In the example of FIG. 1, the lead 112 includes electrodes 124A and 124B (collectively, “electrodes 124”) configured to be positioned on, within, or near cardiac tissue at or near target site 114. In some examples, electrodes 124 provide pacing to heart 122. The electrodes 124 may be electrically connected to conductors (not shown in FIG. 1) extending through the lead body 118. In some examples, the conductors are electrically connected to therapy delivery circuitry of IMD 126, with the therapy delivery circuitry configured to provide electrical signals through the conductor to electrodes 124. Electrodes 124 may conduct the electrical signals to the target tissue of heart 122, causing the cardiac muscle, e.g., of the ventricles, to depolarize and, in turn, contract at a regular interval. Electrodes 124 may also be connected to sensing circuitry of IMD 126 via the conductors, and the sensing circuitry may sense activity of heart 122 via electrodes 124.

The electrodes 124 may have various shapes such as tines, helices, screws, rings, rings of discrete segments, partial rings, coils and so on. Again, although a bipolar configuration of lead 112 including two electrodes 124 is illustrated in FIG. 1, in other examples lead 112 may include different numbers of electrodes, such as one electrode, three electrodes, or four electrodes. In configurations in which the lead is a defibrillation lead, the lead may include one or more defibrillation coil electrodes and respective conductors extending through the lead body. In other examples (not shown in FIG. 1), the IMD 126 can be connected to two leads (atrium and right ventricle) or three leads (A, RV, LV), or other electrode or lead configurations.

The configuration of the therapy system 100 illustrated in FIG. 1 is merely one example. In other examples, a therapy system may include epicardial leads, subcutaneous, substernal, and/or patch electrodes instead of or in addition to the transvenous lead 112 illustrated in FIG. 1. Further, the IMD 126 need not be implanted within the patient 116. In examples in which the IMD 126 is not implanted in the patient 116, the IMD 126 may deliver therapies to the heart 122 via percutaneous leads that extend through the skin of patient 116 to a variety of positions within or outside of heart 122.

In one or more examples, IMD 126 includes electronic circuitry contained within an enclosure where the circuitry may be configured to deliver cardiac pacing. In the example of FIG. 1, the electronic circuitry within IMD 126 may include therapy delivery circuitry electrically coupled to electrodes 124. The electronic circuitry within IMD 126 may also include sensing circuitry configured to sense electrical activity of heart 122 via electrodes 124. The therapy delivery circuitry may be configured to administer cardiac pacing via electrodes 124, e.g., by delivering pacing pulses in response to expiration of a timer and/or in response to detection of the activity (or absence thereof) of the heart.

In some examples, the system 100 includes an optional external device 130 such as a programmer. For example, optional external device 130 can be a handheld computing device such as a tablet or a phone, a computer workstation, or a networked computing device. The optional external device 130 can include a user interface that receives input from a clinician, which can include a keypad and a suitable display such as, for example, a touch screen display, or a peripheral pointing device, such as a mouse, via which a user may interact with the user interface. The clinician may also interact with the external device 130 remotely via a networked computing device.

After the implantable medical lead 112 and the electrodes 124 have been temporarily or more permanently implanted in the heart of a patient, the lead 112 and electrodes 124 may need to be removed due to structural defects, infections, or the need to upgrade a pre-existing system. After implantation for extended periods of time (for example, greater than about 1 year), chronic leads may develop a dense fibrotic and sometimes calcific process within the thin-walled venous structures or the endocardial surface of the heart or tricuspid valve, which can make the lead 112 and electrodes 124 difficult to extract. Complex lead extraction is associated with the risk of vascular injury by traction or perforation, causing tamponade, hemothorax, arteriovenous fistula, tricuspid valve disruption, or possibly pulmonary embolism, so simplifying or otherwise improving lead extraction techniques can have significant value for patient safety.

Extraction of leads can be performed by a variety of techniques, and in many cases simple traction or traction devices can be used to remove the lead from the vein of the patient. However, for chronic leads, various types of extraction sheaths, including mechanical, laser, electrosurgical, rotating threaded tip, and telescoping sheaths may be advanced over the lead and into the vein of the patient to remove the fibrotic scar tissue retaining the lead.

For various types of IMDs 126 (FIG. 1), standard male connector pins of lead 112, such as IS-1, IS-4, DF-4, SQ-1, and the like, have one or more structural features configured to engage a female receptacle in the IMD 126. These standard connector pins have a relatively large diameter compared to the body of the lead 112, and if left intact can require an extraction sheath with a larger diameter to be used. However, as noted above, larger diameter extraction sheaths increase the likelihood of vascular injury, so smaller diameter extraction sheaths are preferred for most patients. Consequently, some extraction techniques include cutting the larger diameter connector pin off of the lead, e.g., lead 112. However, as described above, preparation of the proximal portion of a cut implantable medical lead to receive and withstand necessary extraction force for lead extraction can be complicated and time consuming.

Lead extenders according to the present disclosure may overcome some of the problems associated with preparing the proximal portion of cut implantable medical lead to receive traction force while being usable with a relatively smaller diameter extraction tool. Lead extenders according to the present disclosure may include a connector that defines a chamber configured to receive a proximal portion of a cut implantable medical lead. The connector may include a locking mechanism configured to lock the proximal portion of the cut implantable medical lead to an inner wall of the chamber, e.g., in a manner sufficient to maintain connection of the connector to the implantable medical despite application of traction force during a lead extraction procedure. The lead extender may also include an extension line extending from the connector. The length of the extension line may be sufficient to extend through a lumen of an extraction sheath or tool to a proximal end of the extraction tool. Traction force may be applied to the extension line at or near the proximal end of the extraction tool during a procedure to extract the implantable medical lead using the extraction tool, e.g., as the extraction tool is advanced over the connector and the cut implantable medical lead to the distal portion of the implantable medical lead.

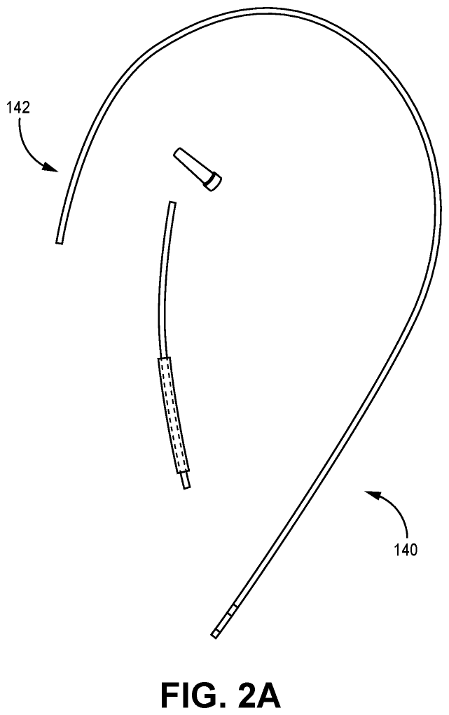

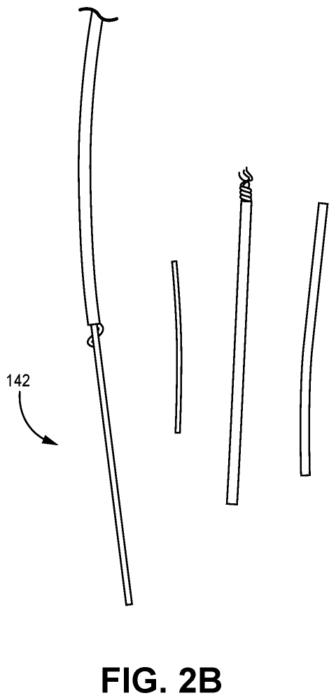

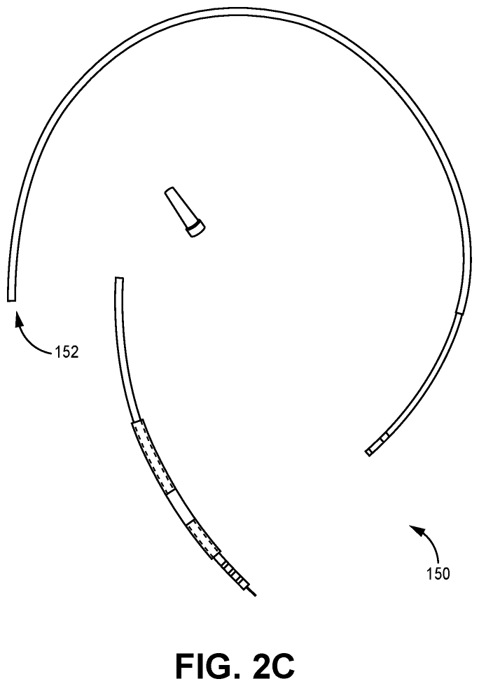

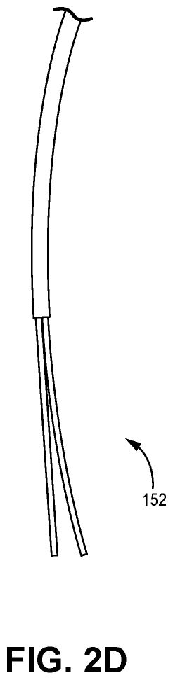

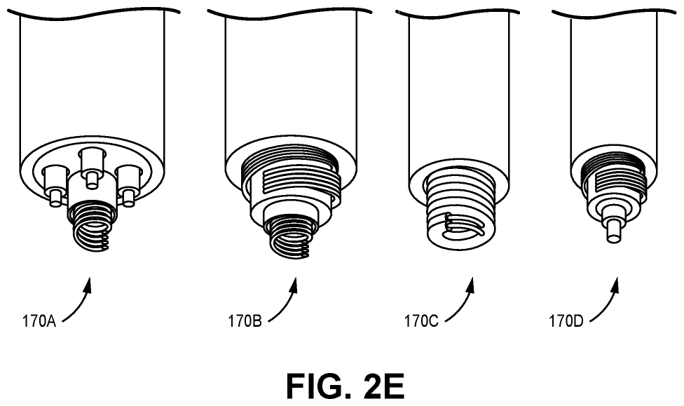

FIGS. 2A-2E illustrate example cut implantable medical leads. For example, FIG. 2A illustrates a bipolar implantable medical lead 140 after a connector has been cut off. Consequently, cut implantable medical lead 140 has a cut proximal portion 142. FIG. 2B illustrates cut proximal portion 142 further prepared by removing some portions of the insulative body and a portion of a conductor coil. As used herein, a proximal portion of a cut implantable medical lead may refer to a proximal portion that has been prepared by removing one or more layers, components, or portions thereof.

FIG. 2C illustrates a multipolar implantable medical lead 150 for use with an implantable cardioverter-defibrillator after a proximal connector has been cut off. FIG. 2D illustrates cut proximal portion 152 further prepared by removing some portions of the insulative body.

FIG. 2E illustrates cut proximal ends 170A-170D (collectively, “cut proximal ends 170”) of a variety of leads having different internal structures. As can be seen in FIG. 2E, once cut and, in some cases, prepared, there are a number of possible lumen and gaps between structures within the lead that may be accessible by a fluid.

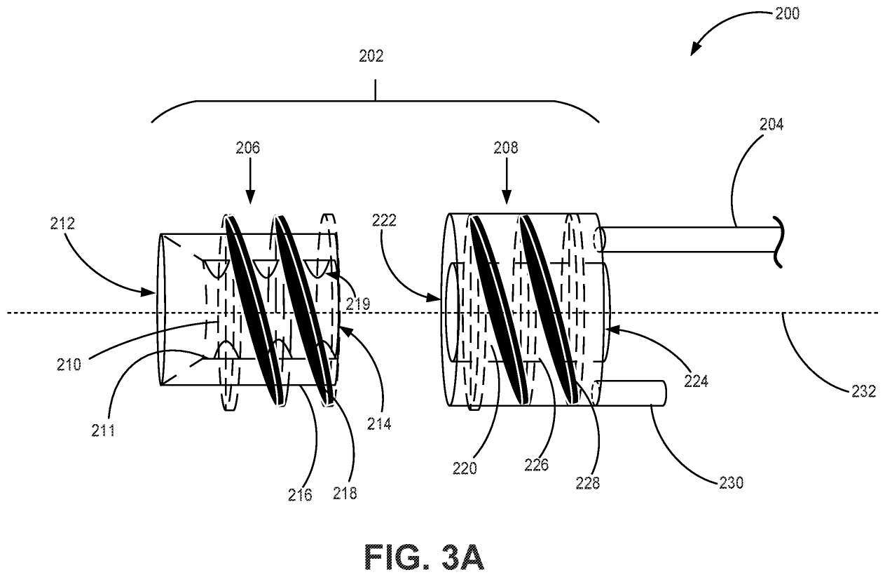

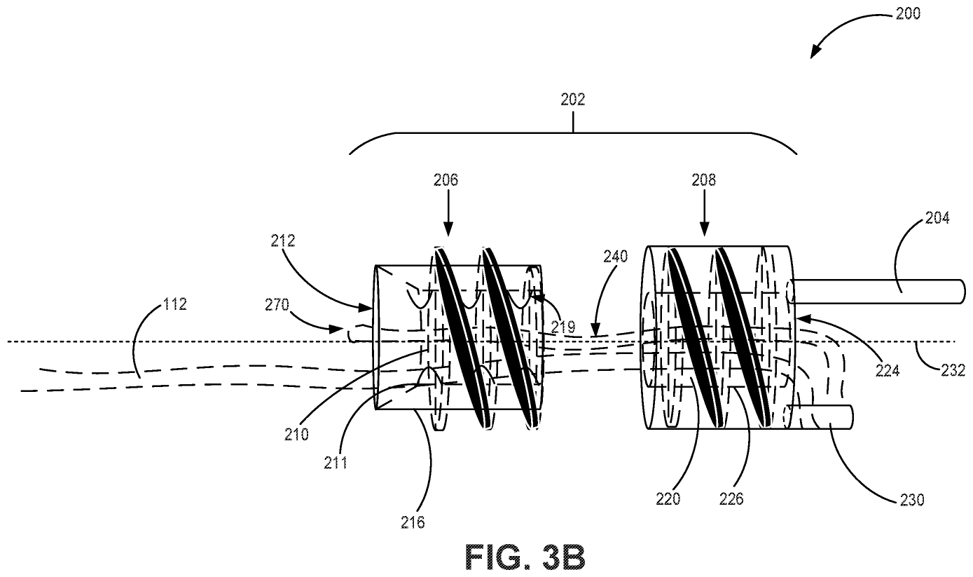

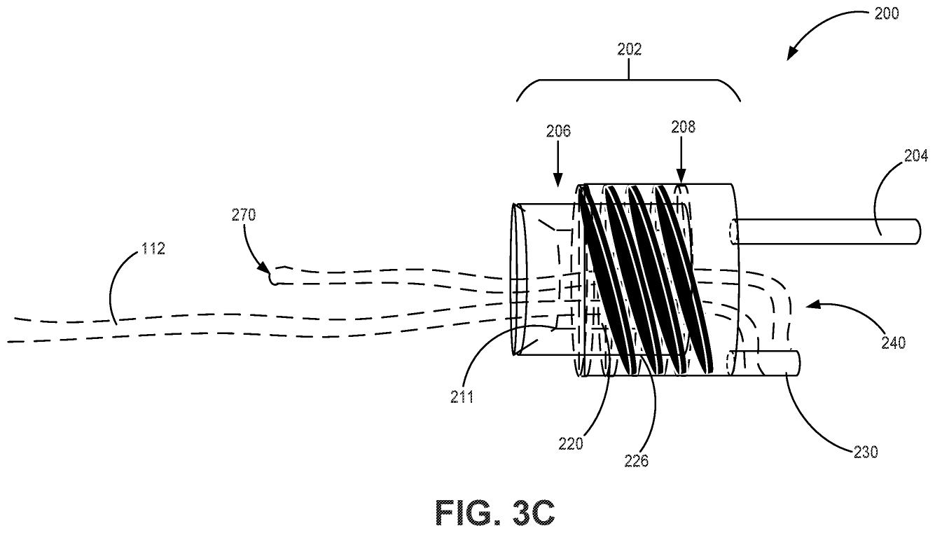

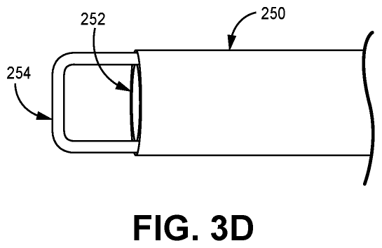

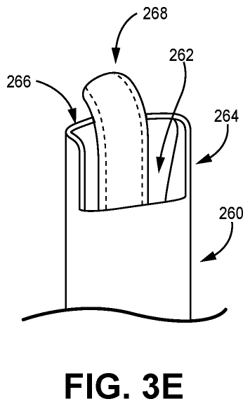

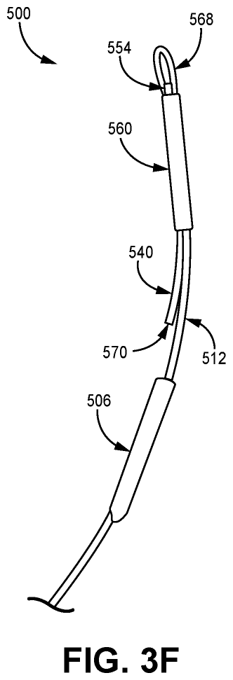

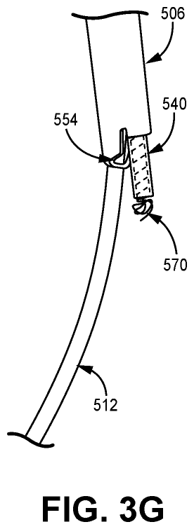

FIGS. 3A-3C are conceptual diagrams illustrating an example lead extender 200, in accordance with one or more techniques of this disclosure. As illustrated in FIG. 3A, lead extender 200 includes a connector 202 and an extension line 204. Connector 202 includes a deformable sleeve 206 and a compression cap 208 that may, as illustrated in FIGS. 3A and 3B, be two separate or separable components. Deformable sleeve 206 comprises a sleeve lumen 210 defined by an inner wall 211. Sleeve lumen 210 extends from a proximal opening 212 to a proximal opening 214. Deformable sleeve 206 further comprises an exterior threaded surface 216 including threads 218.

Connector 202 may define a chamber having inner wall 211 and proximal opening 214 into the chamber, the chamber being configured to receive a proximal portion of a cut implantable medical lead through proximal opening 212, wherein connector 202 comprises a locking mechanism configured to lock the proximal portion of the cut implantable medical lead to the inner wall 211 of the chamber. Extension line 204, having a distal end attached to connector 202, may be configured to extend through a lumen of an extraction tool from a distal end to a proximal end of the extraction tool (not illustrated). In some examples, connector 202 may be configured to move through a lumen of the extraction tool (not illustrated).

Compression cap 208 comprises a compression cap lumen 220 extending from a proximal opening 222 to a distal opening 224. An interior threaded surface 226 comprises thread 228 and defines compression cap lumen 220. Interior threaded surface 226 configured to align with the exterior threaded surface 216 to facilitate threading together of the threaded surfaces. A distal end of extension line 204 is connected to compression cap 204 for extending through a lumen of an extraction tool and application of traction force to connector 202 and, thereby, to the cut implantable medical lead.

When the threaded surfaces are threaded together as shown in FIG. 3C, compression cap lumen 220 may receive at least a portion of deformable sleeve 206. When the threaded surfaces are threaded together, deformable sleeve 206 is configured to deform toward a longitudinal axis 232 of connector 200, when exterior threaded surface 216 and interior threaded surface 226 are threaded together. In some examples, the threading together of the exterior threaded surface 216 and interior threaded surface 226 may reduce the volume of the chamber configured to receive proximal portion 240 of implantable medical lead, such as implantable medical lead 112. The chamber is defined by inner wall 211 and, in some cases, a portion of interior threaded surface 226, and includes sleeve lumen and, in some cases, a portion of compression cap lumen 220. The reduced volume chamber constrains proximal portion 240 of the cut implantable medical lead within the chamber against inner wall 211 to lock the cut implantable medical lead to connector 200. In some examples, as illustrated by FIG. 3A, deformable sleeve 206 may comprise one or more protrusions 219 from inner wall 210 into sleeve lumen 210, which may be configured to pinch, grab, bind, or otherwise interact with proximal portion 240 of the cut implantable medical lead within the chamber when exterior threaded surface 216 and interior threaded surface 226 are threaded together and deformable sleeve 206 is deformed. Protrusions 219 may take any of a variety of forms, such as bumps, spikes, ramps, fingers, or teeth.

In some examples, as illustrated in FIG. 3B, proximal portion 240 of the cut implantable medical lead may be advanced through sleeve lumen 210 and into or through compression cap lumen 220 prior to the threading of the interior and exterior threaded surfaces together to deform deformable sleeve 206. In some examples, proximal portion 240 may be folded prior to the threading of the interior and exterior threaded surfaces together to deform deformable sleeve 206. In some examples, proximal portion 240 may be advanced out of distal opening 224 of compression cap 208 and advanced, wound, or looped around an anchor post 230 of the compression cap to facilitate the folding of proximal portion 240 or advancement of proximal portion 240 in an opposite direction through compression cap lumen 220 and into or through sleeve lumen 210 prior to the threading of the interior and exterior threaded surfaces together to deform deformable sleeve 206. In some examples, proximal portion 240 is folded prior to advancement through lumen 210, 220 and out of distal opening 224, and the loop formed by the folding is placed over post 230. The cut lead may be effectively fixed to anchor post 230 and doubled over itself inside the chamber defined by connector 202.

Anchor post 230 is positioned relative to distal opening 224 of the connector such that proximal portion 240 of the cut implantable medical lead 112 may be advanced through distal opening 224 in a first direction, may be looped around anchor post 230, and back through distal opening 224 in a second direction opposite the first direction. The position and configuration of anchor post 230 is an example, and in other examples, anchor post 230 may have a different position or configuration, such as a cross-bar extending across distal opening 224. Although lumen 210, 220 and the chamber formed when deformable sleeve 206 and compression cap 208 are joined are shown having a relatively uniform diameter along longitudinal axis 232 in the example of FIGS. 3A-3C, in other examples, the chamber may have portions with different diameters, such as narrower diameters near proximal opening 212 of deformable sleeve 206 and distal opening 224 of compression cap 208 to discourage proximal portion 240 from “backing out” of the chamber.

Compression cap 208 may be formed of one or more materials, such as polymer(s) or metal(s), that are relatively more rigid than the one or more materials, such as one or more polymer(s) from which deformable sleeve 206 is formed. Upon threading exterior threaded surface 216 and interior threaded surface 226 together, the relatively more rigid compression cap 208 may deform deformable sleeve 206 and compress the deformable sleeve onto proximal portion 240 of the cut implantable medical lead.

Although described in the context of examples in which deformable sleeve 206 and compression cap 208 have respective threaded surfaces 216, 226, other techniques for coupling deformable sleeve 206 and compression cap 208 and deforming the deformable sleeve may be employed. In some examples, the components may be coupled and the deformable sleeve deformed by a press fit or a snap fit. In some examples, the snap fit may include a rigid or elastomeric keying structure to retain the connection after being overcome by user force to create the connection, such as a spring loaded bump or bayonet, or a deformable lip.

FIGS. 3D and 3E illustrate example configurations of anchor posts that may be used with a lead extender as illustrated in FIGS. 3A-3C. For example, FIG. 3D illustrates an example compression cap 250 including a distal opening 252 and an anchor post arranged relative to distal opening 252. In the example of FIG. 3D, anchor post is arranged as a cross-bar 254 extending across distal opening 252. A distance between distal opening 252 and cross-bar 254 in a longitudinal direction may be greater or less than that illustrated in FIG. 2D. In some examples, cross-bar 254 may be substantially flush with or within distal opening 252.

FIG. 3E illustrates another example compression cap 260 including a distal opening 262 and an anchor post 264 arranged relative to distal opening 262. Like the example of FIG. 3D, in the example of FIG. 3E, anchor post is arranged as a beveled cross-bar 264 across distal opening 262. Beveled cross-bar 264 and distal opening 262 may be formed by removing material from a distal end of compression cap 260. In the illustrated example, beveled cross-bar 264 includes a notch 266 or tapped portion for looping or advancing proximal portion 268 of a cut implantable medical lead around beveled cross-bar 264. Although not illustrated, in some examples deformable sleeve 206 may include an anchor post, cross-bar, or beveled cross-bar similar to any of those illustrated herein arranged relative to proximal opening 212.

FIG. 3F illustrates an example configuration of a lead extender 500 without an extension line, according to this disclosure. Lead extender 500 includes a compression cap 560 and a deformable sleeve 506. Compression cap 560 includes anchor post 554. A proximal portion 540 of cut implantable medical lead 512 is fed through deformable sleeve 506, through compression cap 560 in a first direction, around anchor post 554, and back through compression cap 560 in a second direction opposite the first direction. In some examples, cut end 570 of proximal portion 540 may be further fed back through deformable sleeve 506.

A locking mechanism of some examples may rely of frictional forces induced from feeding cut implantable medical lead 512 around anchor post 554. As in this example the locking mechanism depends on frictional forces induced from wrapping the lead around anchor post 554. In some examples, deformable sleeve 506 and compression cap 560 may be rigid and not deformable, utilizing the locking forces induced from the friction between cut implantable medical lead 512 and an external surface of anchor post 554. When compression cap 560 and deformable sleeve 506 may configured to be mechanically rigid, a single contiguous piece may be used as both compression cap 560 and deformable sleeve 506 (not illustrated). For example, compression cap 560 may form a single contiguous piece with deformable sleeve 506.

FIG. 3G illustrates an example of a deformable sleeve 506, according to this disclosure. Deformable sleeve 506 may include an anchor post 554. Anchor post 554 may be similar or equivalent to any of the previously described anchor posts of this disclosure. Anchor post 554 may be a bar across a distal or proximate opening of deformable sleeve 506. Deformable sleeve 506 may be configured to receive and lock cut implantable medical lead 512.

Proximal portion 540 may be fed into a proximal opening defined by an inner wall of deformable sleeve 506 lumen. The proximal portion may be advanced through the lumen out of a distal opening of the deformable sleeve 506 in a first direction of a first traversal. Implantable medical lead 512 may be configured to traverse a lumen of a compression cap and wrap around a second anchor pin before traversing back through a compression cap as shown in FIG. 3F. Proximal portion 540 may be further fed back through deformable sleeve 506 in a second direction opposite the first direction of the first traversal. Anchor post 554 may be configured to define two proximal openings of deformable sleeve 506, wherein a first opening of the two proximal openings received proximal portion 540 oriented in the first direction and second opening receives cut end 570 of proximal portion 540 oriented in the second direction. In some examples, the first direction may be along a longitudinal access from the first opening to the distal opening and the second direction may be along the longitudinal access from the distal opening to the second opening. Cut end 570 of proximal portion 540 may be advanced to be distal to anchor post 554.

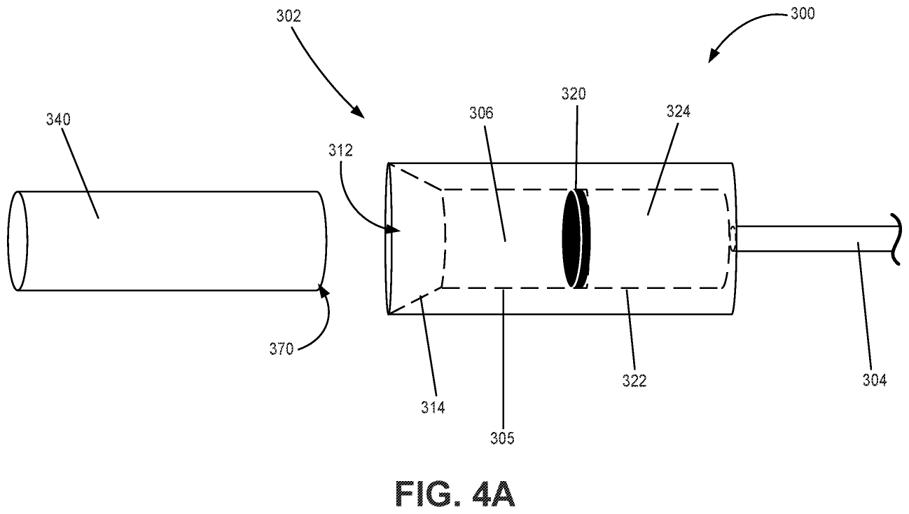

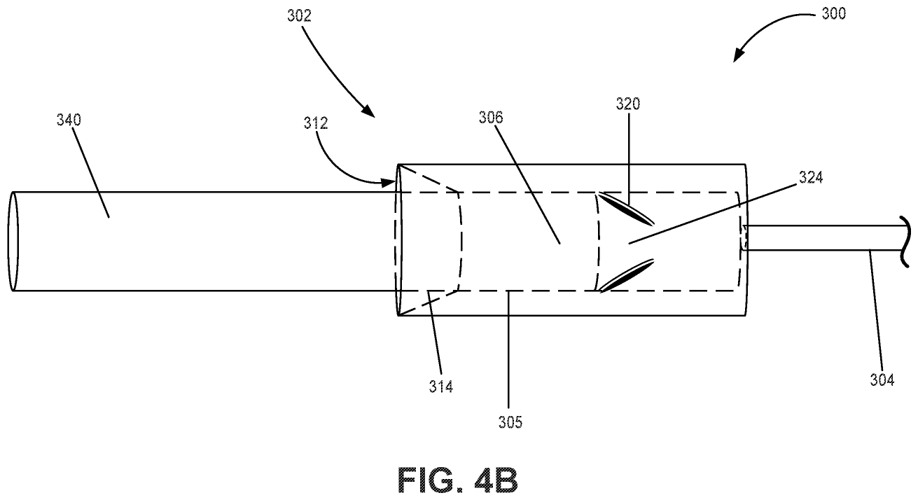

FIGS. 4A and 4B are conceptual diagrams illustrating another example lead extender 300. Lead extender 300 includes a connector 302 and an extension line 304 extending from connector 302, e.g., to extend through a lumen of an extraction tool and apply traction force to connector 302 and, thereby, to a cut implantable medical lead. Connector 302 includes an inner wall 305 that defines a chamber 306 configured to receive a proximal portion 340 of a cut implantable medical lead through an opening 312. Although not illustrated in FIGS. 4A and 4B, proximal portion 340 may be folded and/or twisted onto itself one or more times prior to insertion into chamber 306 through opening 312. Connector 302 also includes a rupturable membrane seal 320 defining an epoxy bladder 322 containing inactivated epoxy 324 within chamber 306, e.g., the deepest or most distal portion of chamber 306.

As illustrated by FIG. 4B, with proximal portion 340 within chamber 306, rupturable membrane seal 320 is ruptured to release epoxy 324 from bladder 322 throughout chamber 306. Membrane seal 320 may be ruptured in response to or after insertion of proximal end 340 into chamber 306. In some examples, membrane seal 320 is configured to rupture in response to application of force to connector 302 by a user. In some examples, membrane seal 320 is configured to rupture by a cut end of the implantable medical lead inserted into chamber 306.

For example, connector 302 or a portion thereof may be deformable, and the user may pinch or squeeze connector 302 or the portion to rupture membrane seal 320, e.g., by reducing a volume of bladder 322 and increasing pressure of epoxy 324 therein. In some examples, membrane seal 320 is configured to be ruptured by proximal end 340 when advanced into chamber 306.

Epoxy 324 may be activated, e.g., cured, after or in response to rupturing membrane seal 320 to lock proximal portion 340 of the cut implantable medical lead to inner wall 305 of chamber 306. Epoxy 324 may be configured to cure within a few seconds to a few minutes of the rupturing of membrane seal 320. Epoxy 324 may be activated or cured by ultraviolet light, oxygen, or other know techniques. If the epoxy is activated through exposure to oxygen, the epoxy will start curing once the membrane ruptures and the epoxy is in contact with air. Activated epoxy 324 provides a rigid connection of proximal portion 340 to connector 302 to allow application of traction force to the implantable medical lead via extension line 404.

As illustrated in FIGS. 4A and 4B, a thickness of inner wall 305 may be tapered at a portion 314 proximate to opening 312. When proximal portion 340 of the cut implantable medical lead is inserted through opening, tapered thickness portion may apply back suction to epoxy 324 pulling the epoxy proximally through chamber 306 and along proximal portion 340. In some examples, the back suction may force epoxy 324 into lumen and/or other spaces within the implantable medical lead that were exposed by cutting the implantable medical lead. Epoxy 324 may have a relatively lower viscosity prior to activation.

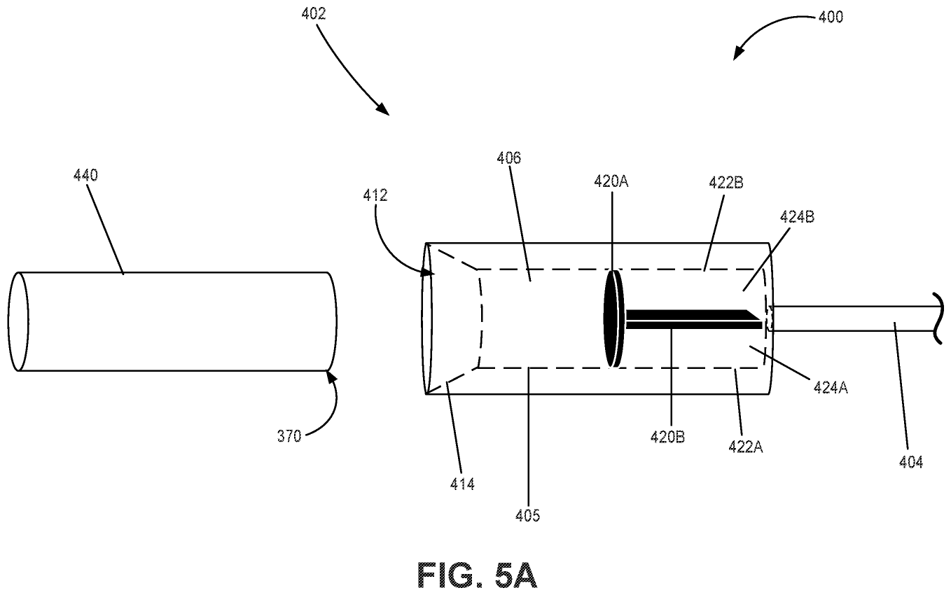

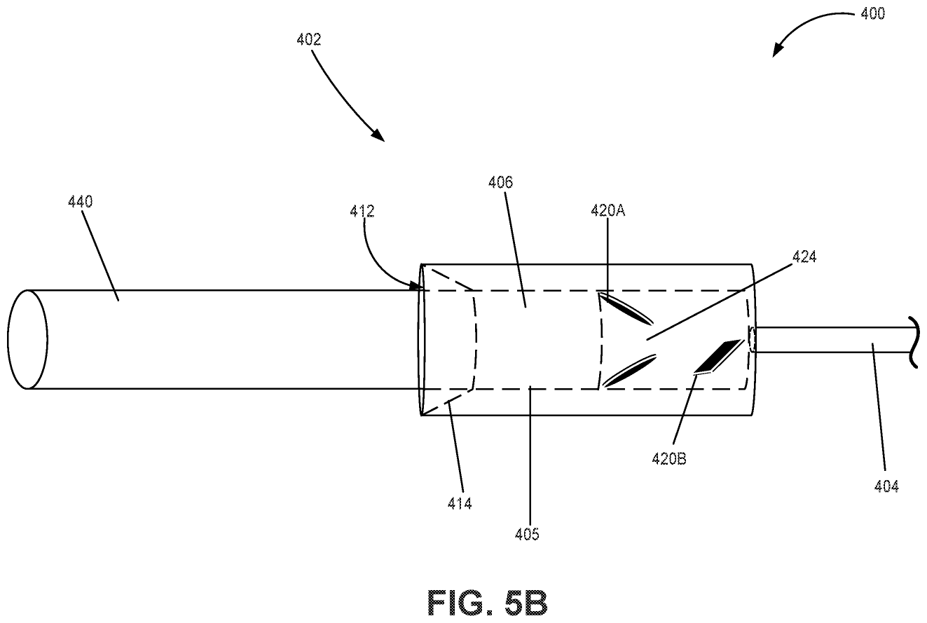

FIGS. 5A and 5B are conceptual diagrams illustrating another example lead extender 400. Lead extender 400 of FIGS. 5A and 5B may be substantially similar to lead extender 300 of FIGS. 4A and 4B except as noted herein. Elements of lead extender 400 may be substantially similar to like numbered elements of lead extender 300 except as noted herein. Similar to lead extender 300, lead extender 400 may include a connector 402 having an inner wall 405 defining a chamber 406 configured to receive a proximal portion 440 of a cut implantable medical lead, and extension line 404 extending from connector 402.

Connector 402 includes a plurality (e.g., one or more) of rupturable membrane seals 420A and 420B (collectively, “rupturable membrane seals 420”) defining a plurality (e.g., one or more) of epoxy bladders 422A and 422B (collectively, “epoxy bladders 422”). Epoxy bladders 422 may be defined within chamber 406. In some examples, an inactivated epoxy may be contained within a plurality of epoxy bladders 422. The inactivated epoxy may be activated in response to rupturing one or more rupturable membrane seals 420, locking a proximal portion of a cut implantable medical lead to inner wall 405 of chamber 406. In some examples, Epoxy bladders 422A and 422B contain respective isolated and inactive epoxy parts 424A and 424B of a multi-part epoxy 424. When membrane seals 420 are ruptured, as shown in FIG. 5B, epoxy parts 424A and 424B mix and are cured or otherwise activated by being mixed.

While inactive when isolated, the epoxy parts 424A and 424B maintain a reasonably low viscosity. However, upon combining, the epoxy parts 424A and 424B mix to form an activated epoxy 424 that begins to cure. While this example describes the use of a two-part epoxy, any number of inactivated epoxy chemical compounds in respective bladders may be used to create a mixable solution resulting in an activated epoxy.

In some examples, an epoxy 324 or 424 may be included with a connector 202 as illustrated in FIGS. 3A-3C. For example, one or both of exterior threaded surface 216 or interior threaded surface 226 may include one or more epoxy bladders 322 or 422 with a respective one or more rupturable membrane seals 320 or 420. The seals may be configured to rupture when the interior and exterior threaded surfaces are threaded together to release the epoxy. The epoxy may strengthen the bond between deformable sleeve 306 and compression cap 308 and/or enter the chamber to bind the proximal portion of the cut implantable medical lead to the inner wall of the connector within the chamber.

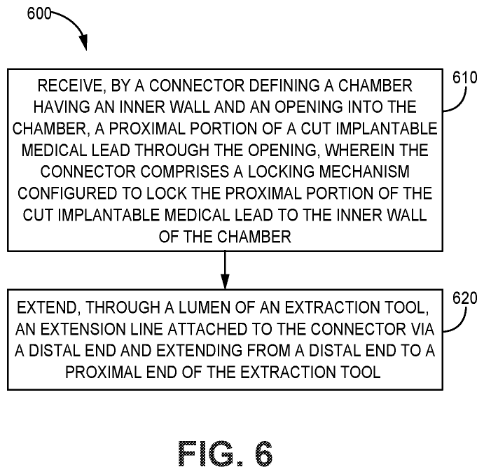

FIG. 6 is a flow diagram illustrating an example method 600 for using a lead extender according to this disclosure. According to the example method of FIG. 6, a clinician may cause a lead extender to receive, by a connector defining a chamber having an inner wall and an opening into the chamber, a proximal portion of a cut implantable medical lead through the opening, wherein the connector comprises a locking mechanism configured to lock the proximal portion of the cut implantable medical lead to the inner wall of the chamber (610). In some examples, the locking mechanism may include a deformable sleave 206 having protrusions 219 of FIG. 3A-3B that grip proximal portion 240 upon deformation. In some examples, the locking mechanism may include epoxy activated by rupturing one or more membranes any of membrane seals 320 or reputable membrane seals 420 of FIG. 4A-4B and FIG. 5A-5B. In some examples, the lock mechanism may include a frictional force induced by looping proximal portion around an anchor post 230 of FIG. 3A-FIG. 3C. In some examples, to cause a connector to receive a proximal portion, a clinician may insert a proximal portion of a cut implantable medical lead through the opening defined by an inner wall of the connector and activate a locking mechanism of the connector to lock the proximal portion of the cut implantable medical lead to the inner wall of the chamber. In various examples, a clinician may activate a locking mechanism by advancing, in a first direction, the proximal portion of the cut implantable medical lead through the distal opening, around the anchor post, and back through the distal opening in a second direction, opposite the first direction.

The clinician may extend, through a lumen of an extraction tool, an extension line attached to the connector via a distal end and extending from a distal end to a proximal end of the extraction tool (620). In some examples, the connector may be any of connector 202, 302, or 402 of FIG. 3A-3C, FIG. 4A-4B, and FIG. 5A-5B. The connector may be configured to move through the lumen of the extraction tool upon the application of traction force along the extension line by a clinician. In various examples, to extend an extension line, a clinician may advance an extension line having a distal end attached to the connector through a lumen of the extraction tool. The clinician may than advance the extraction tool over the connector and the cut implantable medical lead and extract the cut implantable medical lead from a patient using the extraction tool.

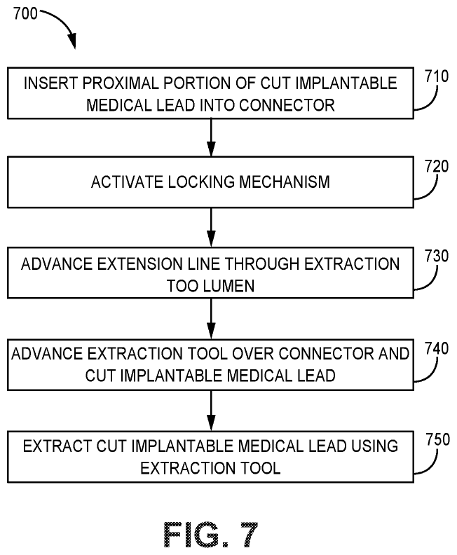

FIG. 7 is a flow diagram illustrating an example method 700 for extracting a cut implantable medical lead using a lead extender according to this disclosure. According to the example method of FIG. 7, a clinician inserts a proximal portion of a cut implantable medical lead into a connector of a lead extender (710). In some examples, the clinician folds or twists the proximal portion on itself one or more times prior to (or by) inserting the proximal portion into the connector. In some examples, the cut lead may be effectively doubled over itself prior to (or by) inserting the proximal portion into the connector. In some examples, inserting the proximal portion into the connector comprises inserting the proximal portion into a chamber 306, 406 defined by an inner wall 305, 405 of the connector. In some examples, inserting the proximal portion into the connector comprises inserting the proximal portion through lumen 210, 220 of deformable sleeve 206 and compression cap 208 and, in some examples, looping proximal portion around an anchor post 230 of compression cap 208.

With the proximal portion of the cut implantable medical lead within the connector, the clinician may activate a locking mechanism to lock the proximal portion within a chamber and/or to an inner wall of the connector (720). In some examples, activating a locking mechanism comprises threading compression cap 208 over deformable sleeve 206 to deform the deformable sleeve, reducing the volume of a chamber containing the proximal portion. In some examples, activating a locking mechanism comprises releasing and activating (curing) an epoxy within the chamber of the connector.

The clinician may advance an extension line extending from the connector through a lumen of a lead extraction tool such that the traction force may be applied to the cut implantable medical lead via the extension line from a proximal end of the lead extraction tool (730). The clinician may then advance the extraction tool over the connector and the cut implantable medical lead, e.g., while the traction force is applied via the extension line connected to the connector of the lead extender (740). Because the proximal portion of the cut implantable medical lead is locked to/within connector, the clinician may apply traction force to the cut implantable medical lead via the extension line while advancing and using the lead extraction tool. The clinician may extract the cut implantable medical lead using the extraction tool (750).

The following numbered examples may illustrate one or more aspects of this disclosure:

Example 1. A lead extender comprising: a connector defining a chamber having an inner wall and an opening into the chamber, the chamber configured to receive a proximal portion of a cut implantable medical lead through the opening, wherein the connector comprises a locking mechanism configured to lock the proximal portion of the cut implantable medical lead to the inner wall of the chamber; and an extension line, having a distal end attached to the connector, the extension line configured to extend through a lumen of an extraction tool from a distal end to a proximal end of the extraction tool.

Example 2. The lead extender of example 1 wherein the connector is configured to move through the lumen of the extraction tool.

Example 3. The lead extender of examples 1 or 2, wherein the connector comprises one or more rupturable membrane seals defining one or more epoxy bladders within the chamber, wherein the locking mechanism comprises an inactivated epoxy contained within the one or more epoxy bladders, and wherein the epoxy is activated in response rupturing the one or more membrane seals to lock the proximal portion of the cut implantable medical lead to the inner wall of the chamber.

Example 4. The lead extender of claim 3, wherein each of the one or more epoxy bladders contains a respective isolated part of a multi-part epoxy.

Example 5. The lead extender of any one or more of claims 3 to 4, wherein the opening further comprises a tapered wall thickness configured to apply back suction to the epoxy when the proximal portion of the cut lead is inserted through the opening.

Example 6. The lead extender of any one or more of claims 3 to 7, wherein the one or more membranes are configured to rupture in response to application of force to the connector by a user.

Example 7. The lead extender of any one or more of claims 3 to 8, wherein the one or more membranes are configured to be ruptured by the cut end of the implantable medical lead inserted into the chamber.

Example 8. The lead extender of claims 1 to 7, wherein the connector comprises: a deformable sleeve comprising a sleeve lumen and an exterior threaded surface; and a compression cap comprising an interior threaded surface configured to align with the exterior threaded surface of the deformable sleeve and defining a compression cap lumen configured to receive at least a portion of the deformable sleeve when the exterior and interior threaded surfaces and threaded together, wherein the opening comprises an opening of the sleeve lumen, the inner wall comprises an inner wall of the sleeve lumen, and the chamber comprises the sleeve lumen, wherein the deformable sleeve is configured to deform toward a longitudinal axis of the connector when the exterior and interior threaded surfaces are threaded together, wherein the locking mechanism comprises the deformable sleeve deformed toward the longitudinal axis to reduce a volume of the chamber and constrain the proximal portion of the cut implantable medical lead to the inner wall within the chamber, and wherein the distal end of the extension line is attached to the compression cap.

Example 9. The lead extender of claims 1 to 8, wherein the locking mechanism comprises an anchor post positioned relative to a distal opening of the connector such that the proximal portion of the cut implantable medical lead may be advanced through the distal opening in a first direction, around the anchor post, and back through the distal opening in a second direction, opposite the first direction.

Example 10. The lead extender of claim 9, wherein the anchor post is a bar across the distal opening.

Example 11. The lead extender of any one or more of claims 1 to 10, wherein at least one of the interior and exterior threaded surfaces further comprises a compression activated epoxy bladder configured to release epoxy when the interior and exterior threaded surfaces are threaded together.

Example 12. The lead extender of claims 8 to 11, wherein the deformable sleeve further comprises one or more protrusions from the inner wall of the deformable sleeve into the sleeve lumen.

Example 13. The lead extender of claims 10 to 12, wherein the cross-bar may be a beveled cross-bar including a notch or tapered portion for looping or advancing the proximal portion of the cut implantable medical lead around the cross-bar.

Example 14. The lead extender of claims 9 to 13, wherein the locking mechanism relies on frictional forces induced from feeding the cut implantable medical lead around the anchor post.

Example 15. The lead extender of claim 8, wherein the compression cap may form a single contiguous piece with deformable sleeve.

1. A lead extender comprising:
a connector defining a chamber having an inner wall and an opening into the chamber, the chamber configured to receive a proximal portion of a cut implantable medical lead through the opening, wherein the connector comprises a locking mechanism configured to lock the proximal portion of the cut implantable medical lead to the inner wall of the chamber; and
an extension line, having a distal end attached to the connector, the extension line configured to extend through a lumen of an extraction tool from a distal end to a proximal end of the extraction tool.

2. The lead extender of claim 1 wherein the connector is configured to move through the lumen of the extraction tool.

3. The lead extender of claim 1, wherein the connector comprises one or more rupturable membrane seals defining one or more epoxy bladders within the chamber, wherein the locking mechanism comprises an inactivated epoxy contained within the one or more epoxy bladders, and wherein the epoxy is activated in response to rupturing the one or more membrane seals to lock the proximal portion of the cut implantable medical lead to the inner wall of the chamber.

4. The lead extender of claim 3, wherein each of the one or more epoxy bladders contains a respective isolated part of a multi-part epoxy.

5. The lead extender of claim 3, wherein the opening further comprises a tapered wall thickness configured to apply back suction to the epoxy when the proximal portion of the cut lead is inserted through the opening.

6. The lead extender of claim 3, wherein the one or more membranes are configured to rupture in response to application of force to the connector by a user.

7. The lead extender of claim 3, wherein the one or more membranes are configured to be ruptured by the cut end of the implantable medical lead inserted into the chamber.

8. The lead extender of claim 1, wherein the connector comprises:
a deformable sleeve comprising a sleeve lumen and an exterior threaded surface; and
a compression cap comprising an interior threaded surface configured to align with the exterior threaded surface of the deformable sleeve and defining a compression cap lumen configured to receive at least a portion of the deformable sleeve when the exterior and interior threaded surfaces and threaded together,
wherein the opening comprises an opening of the sleeve lumen, the inner wall comprises an inner wall of the sleeve lumen, and the chamber comprises the sleeve lumen,
wherein the deformable sleeve is configured to deform toward a longitudinal axis of the connector when the exterior and interior threaded surfaces are threaded together,
wherein the locking mechanism comprises the deformable sleeve deformed toward the longitudinal axis to reduce a volume of the chamber and constrain the proximal portion of the cut implantable medical lead to the inner wall within the chamber, and
wherein the distal end of the extension line is attached to the compression cap.

9. The lead extender of claim 1, wherein the locking mechanism comprises an anchor post positioned relative to a distal opening of the connector such that the proximal portion of the cut implantable medical lead may be advanced through the distal opening in a first direction, around the anchor post, and back through the distal opening in a second direction, opposite the first direction.

10. The lead extender of claim 9, wherein the anchor post is a cross-bar extending across the distal opening.

11. The lead extender of claim 1, wherein at least one of the interior and exterior threaded surfaces further comprises a compression activated epoxy bladder configured to release epoxy when the interior and exterior threaded surfaces are threaded together.

12. The lead extender of claim 1, wherein the deformable sleeve further comprises one or more protrusions from the inner wall of the deformable sleeve into the sleeve lumen.

13. The lead extender of claim 10, wherein the cross-bar may be a beveled cross-bar including a notch or tapered portion for looping or advancing the proximal portion of the cut implantable medical lead around the cross-bar.

14. The lead extender of claim 9, wherein the locking mechanism relies on frictional forces induced from feeding the cut implantable medical lead around the anchor post.

15. The lead extender of claim 8, wherein the compression cap may form a single contiguous piece with the deformable sleeve.

16. A lead extender comprising:
a deformable sleeve comprising a sleeve lumen defined by an inner wall and an exterior threaded surface;
a compression cap comprising an interior threaded surface configured to align with the exterior threaded surface of the deformable sleeve and defining a compression cap lumen configured to receive at least a portion of the deformable sleeve when the exterior and interior threaded surfaces and threaded together; and
an extension line, having a distal end attached to an exterior surface of the compression cap, the extension line configured to extend through a lumen of an extraction tool from a distal end to a proximal end of the extraction tool,
wherein a chamber comprising at least the sleeve lumen is configured to receive a proximal portion of a cut implantable medical lead when the exterior and interior threaded surfaces and threaded together, and
wherein the deformable sleeve is configured to deform toward a longitudinal axis of the connector when the exterior and interior threaded surfaces are threaded together to reduce a volume of the chamber and constrain the proximal portion of the cut implantable medical lead to the inner wall within the chamber.

17. The lead extender of claim 16, wherein the compression cap comprises a distal opening configured to receive the proximal portion of a cut implantable medical lead therethrough, and
wherein the compression cap comprises an anchor pin positioned relative to the distal opening such that the proximal portion of the cut implantable medical lead may be advanced through the distal opening in a first direction, around the anchor pin, and back through the distal opening in a second direction, opposite the first direction.

18. A method comprising:
inserting a proximal portion of a cut implantable medical lead through an opening of a connector and into a chamber defined by an inner wall of the connector;
activating a locking mechanism of the connector to lock the proximal portion of the cut implantable medical lead to the inner wall of the chamber;
advancing an extension line having a distal end attached to the connector through a lumen of an extraction tool;
advancing the extraction tool over the connector and the cut implantable medical lead; and
extracting the cut implantable medical lead from a patient using the extraction tool.

19. The method of claim 18, wherein activating the locking mechanism comprises rupturing one or more rupturable membrane seals defining one or more epoxy bladders within the chamber to release an epoxy to lock the proximal portion of the cut implantable medical lead to the inner wall of the chamber.

20. The method of claim 18, wherein the connector comprises:
a deformable sleeve comprising a lumen defined by the inner wall and configured to receive a proximal portion of a cut implantable medical lead through the lumen, the deformable sleeve further comprising an exterior threaded surface; and
a compression cap having an interior threaded surface defining chamber, the interior threaded surface configured to align with the exterior threaded surface of the deformable sleeve, wherein the deformable sleeve is configured to deform toward a longitudinal axis of the connector when the exterior threaded surface is screwed into the compression cap,
wherein activating the locking mechanism comprises screwing the deformable sleeve and the compression cap together to deform the deformable sleeve toward the longitudinal axis to lock the proximal portion of the cut implantable medical lead to the inner wall.
